(19) United States
(12) Patent Application Publication
(10) Pub. No.: US 2010/0216999 A1
(43) Pub. Date: Aug. 26, 2010

(54) PROCESS FOR PREPARATION OF PURE POLYMORPHIC FORM 1 OF CLOPIDOGREL HYDROGENSULFATE

(76) Inventors: Tomasz Kozluk, Warszawa (PL); Robert Wozniak, Wyszkow (PL)

Correspondence Address: MATTHIAS SCHOLL, 14781 MEMORIAL DRIVE, SUITE 1319, HOUSTON, TX, 77079, US

(21) Appl. No.: 12/531,888

(22) PCT Filed: Mar. 19, 2008
(86) PCT No.: PCT/PL08/00023

§ 371 Date: Feb. 25, 2010

(30) Foreign Application Priority Data
Mar. 23, 2007 (PL) P-382055

(51) Int. Cl. C07D 513/04

(52) U.S. Cl. 546/114

(57) ABSTRACT

Process for the preparation of substantially pure polymorphic Form 1 of clopidogrel hydrogensulfate, wherein the reaction of optically active clopidogrel base with sulfuric acid is carried out in the mixture of at least two solvents, chosen from group I, comprising aliphatic ethers, and from group II, comprising ketones, esters of C₁-C₃ carboxylic acids and aliphatic alcohols C₁-C₄, primary, secondary and tertiary aliphatic alcohols C₁-C₄, then the resulting suspension is stirred until at least 70% of amorphous clopidogrel hydrogensulfate formed is transformed into polymorphic Form 1 of clopidogrel hydrogensulfate, and finally, the crystalline solid is isolated from the reaction mixture and subject to additional work-up procedure, aiming at the formation and stabilization of polymorphic Form 1.

### FIELD OF THE INVENTION

The invention relates to the preparation of substantially pure polymorphic Form 1 of clopidogrel hydrogensulfate.

Clopidogrel, methyl(+)-(S)-α-(2-chlorophenyl)-4,5,6,7-tetrahydrothieno[3,2,-c]pyridin-5-acetate, is an antiplatelet agent. This compound is commercially available under the trade name Plavix®, for platelets aggregation inhibition and reduction of blood coagulability.

### BACKGROUND OF THE INVENTION

The process for preparation of pharmacologically active dextrorotatory clopidogrel S-isomer has been disclosed in the European Patent EP 0281459 B1. The process described therein is based on the successive crystallization of methyl(+)-(S)-α-(2-chlorophenyl)-4,5,6,7-tetrahydrothieno[3,2,-c]pyridin-5-acetate diastereoisomeric salts with optically active acid, such as 10-camphorsulfonic acid, until constant optical rotation value is reached. Upon treatment of the diastereoisomeric salts with a base, S-(+)-clopidogrel isomer is being released. This isomer is subsequently converted into hydrogensulfate salt in the reaction with 80% sulfuric acid. Both processes, the salt crystallization with 10-camphorsulfonic acid as well as hydrogensulfate salt formation occur in acetone. Isolated this way S-(+)-clopidogrel hydrogensulfate, characterized by melting point 184° C. and optical rotation [α]²⁰_{D}=+55,10° in methanol, is referred as the polymorphic Form 1.

From the International Patent Application WO 99/65915 it is known that clopidogrel hydrogensulfate can also exist in the thermodynamically more stable polymorphic Form 2 obtained during the process of long-term crystallization, 3 to 6 months, from the aqueous-acetone mother liquor left from polymorphic Form 1 crystallization. This mother liquor containing up to 10% clopidogrel hydrogensulfate and from 0.3 to 1% water, was maintained at temperature 40° C.

In the Polish Patent Application No. P-355814 amorphous form of clopidogrel hydrogensulfate has been described. Amorphous solid is formed when the reaction of optically active base methyl(+)-(S)-α-(2-chlorophenyl)-4,5,6,7-tetrahydrothieno[3,2,-c]pyridin-5-acetate with sulfuric acid is carried out in tert-butyl methyl ether with certain amount of isopropyl alcohol, preferably 0.5-2 volume parts of alcohol per 20 volume parts of ether. It was experimentally proved, that amorphous clopidogrel hydrogensulfate is formed at temp. 0-5° C. from diluted solutions (about 3% wt of clopidogrel free base in 1 L of solvent), after 1-5 h since the completion of sulfuric acid dropping.

The International Patent Application WO 2004/048385 discloses that the increased concentration of optically active clopidogrel free base in the solution and longer stirring time of the forming hydrogensulfate salt in tert-butyl methyl ether, promote the crystallization of clopidogrel hydrogensulfate polymorph 1. In the same patent application, the altered preparation method of high enantiomeric purity polymorph 1 is also described. This method is based on the precipitation of the clopidogrel salt, obtained in the reaction of optically active clopidogrel base and concentrated sulfuric acid in a specific aliphatic or cyclic ether, such as: dimethoxyethane, diethoxyethane, tert-butyl methyl ether, bis-2-ethoxyethyl ether and dioxane, as well as isobutyl methyl ketone. US 2003/114479 A1 discloses that the dispersion of amorphous clopidogrel hydrogensulfate transforms into polymorphic form 1 upon stirring in tert-butyl methyl ether. Among preferable ethers, tert-butyl methyl ether and diethyl ether are mentioned. According to the experiments performed, amorphous clopidogrel hydrogensulfate transforms into polymorph 1 while stirred in the ether solution for 45 min. to 1 hour; as it is said in the description, preferable stirring time is from 4 to 8 hours.

WO 03/051362 reveals the other clopidogrel hydrogensulfate polymorphs. The Inventors claim clopidogrel hydrogensulfate solvates with 1-butanol (Form III), with isopropanol (Form IV), with 2-butanol (Form V) and with 1-propanol (Form VI). Following several experiments, except the reaction carried out in isopropanol, clopidogrel hydrogensulfate salts isolated from the mentioned alcohols and dried, prove to be polymorphs 1 and 2. In none of the experiments the solvate formation was observed.

In the International Patent Application WO 2004/020443 the product resulting from the reaction of clopidogrel base and sulfuric acid is isolated from primary, secondary or tertiary alcohols C₁-C₅, their esters with C₁-C₄ carboxylic acids or the mixtures thereof.

In WO 2005/100364 the reaction of clopidogrel base with sulfuric acid, either concentrated or diluted, is performed in the ether solution (diethyl ether, diisopropyl ether, tert-butyl methyl ether) lower ester, methylene chloride or the mixtures thereof, in temperature range from −15° C. to +5° C. After sulfuric acid dropping completion, the mixture is stirred for additional 10 h in temperature range −10° C. to +10° C., the resulting crystalline solid is filtered off, washed and dried, yielding polymorphic Form 1.

In WO 2005/104663 concentrated sulfuric acid is dropwise added to clopidogrel base solution in methyl propyl ketone, methyl isopropyl ketone or diethyl ketone, the mixtures thereof, or in the ketones and ethyl acetate mixtures in the temperature range −10° C. to +20° C., reaction is maintained at 28-30° C. for 7 to 10 h, until the precipitation of polymorphic Form 1 occurs.

According to US 2006/0183907 A1, the reaction of clopidogrel base with concentrated sulfuric acid is performed in ethyl acetate, with crystallization seeded with polymorph 1 crystals. The resulting suspension is stirred for 1 h at reflux and for another 1 h at room temperature.

In WO 2006/087729 clopidogrel base is dissolved in a solubilizing solvent, preferably in acetic acid, to this solution sulfuric acid in aliphatic ether, eg., diisopropyl ether, is added. From this solvents mixture polymorph 1 precipitates out.

According to US 2006/0205766 A1, polymorphic Form 1 of clopidogrel hydrogensulfate is obtained in the reaction of clopidogrel base with sulfuric acid in 2-propanol or 2-butanol, followed by crystallization seeding with appropriate crystals. The mixture is stirred at temp. 25-30° C. and maintained at the same temperature for 1 h. When the crystalline solid is filtered off, second crop of polymorph 1 is collected from the mother liquor. From the filtrate left for indefinite period of time, the mixture of polymorphs 1 and 2 precipitates.

It has also been proved (EP 1693375 A1), that amorphous clopidogrel hydrogensulfate transforms into polymorph 1 upon stirring of the suspension in alkane C₆₋₁₀ for 40 h at temperature not lower that 30° C.

Until now polymorphic Form 1 of clopidogrel hydrogensulfate was generally obtained, by dropping concentrated sulfuric acid into clopidogrel base solution and subsequent isolation of the precipitated solid after indefinite period of time. Solvents described so far, from which clopidogrel hydrogensulfate polymorphic Form 1 precipitates are as follows: aliphatic and cyclic ethers, lower asymmetric and symmetric ketones, and esters of lower carboxylic acids and aliphatic alcohols C₁₋₄.

The difficulties regarding isolation of substantially pure clopidotrel hydrogensulfate polymorphic Form 1, resulted from the fact, that in the same solvent systems, the transformation of amorphous hydrogensulfate salt into polymorph 1 occurs, followed by the transformation of polymorph 1 into thermodynamically more stable polymorph 2. The rate of this transformation is unpredictable, because it depends not only on the type of solvent used, but also on its purity and the reaction conditions, like for example: reaction mixture dilution, temperature, etc. In addition, when concentrated sulfuric acid is used, the enantiomeric purity of the obtained product is decreased. Methods applied so far are not effective for the selective preparation of clopidogrel hydrogensulfate polymorphic Form 1, and in consequence the isolated polymorph 1 can be contaminated with some amounts of amorphous or/and polymorph 2 by-products. This phenomenon may be troublesome, especially in the plant production process. If the production batch consists of non-homogenous product, it is required to release clopidogrel base from its salt and return it for further crystallization process. These additional operations are necessary, in order to obtain product, which meets pharmaceutical substance (API) specifications. Thermodynamic transformation may also occur in the isolated product during its handling or storage.

There is still the need to develop an easy method to control clopidogrel hydrogensulfate preparation and isolation, which enables formation of the stable polymorph 1 crystals, deprived of other crystalline forms contaminations. The new process parameters are to meet some requirements; reaction time shouldn't be exceedingly long and the number of process operations connected with product recrystallization should be minimal.

### DESCRIPTION OF THE FIGURES

FIG. 1 presents IR spectra of the following compounds: clopidogrel hydrogensulfate polymorphic Form 1, prepared by the process of the invention, reference polymorphic Form 2 and clopidogrel amorphous form, measurement range 400.0-4000.0 cm⁻¹.

FIG. 2 presents X-ray powder diffraction pattern of clopidogrel hydrogensulfate polymorphic Form 1 prepared by the process of the invention.

### ABBREVIATIONS USED

MIBK—methyl isobutyl ketone
MTBE—tert-butyl methyl ether
ETBE—tert-butyl ethyl ether
EA—ethyl acetate

### DISCLOSURE OF THE INVENTION

The research results of clopidogrel hydrogensulfate crystallization kinetics brought us to the conclusion, that the solvent dependent transformation rates of amorphous form into polymorphic Form 1 and, subsequently, from polymorphic Form 1 into polymorphic Form 2, could be controlled by changing the composition of the solvents mixture used. This method enables preparation of substantially crystalline pure clopidogrel hydrogensulfate polymorphic Form 1, deprived of other polymorphic forms impurities, highly stable and not susceptible to further transformations.

The term ‘substantially pure polymorphic Form 1’ used hereafter, is defined as the free of other polymorphic form impurities clopidogrel hydrogensulfate substance. These impurities can be present in the amounts undetectable by routinely used analytical methods, such as: X-ray powder diffraction and IR spectroscopy. It means, the contamination with other polymorphic forms is less than 2%, preferably less than 1%. Substantially pure polymorphic Form 1 of clopidogrel hydrogensulfate contains total chemical impurities less than 0.1% and its optical purity is 99.5% or higher. In the embodiment of the present invention, the preparation of substantially pure polymorphic Form 1 of clopidogrel hydrogensulfate is accomplished in the reaction of clopidogrel base and sulfuric acid in the carefully selected solvent systems. The proper crystallization solvent systems are those in which the crystallization of polymorphic Form 1 proceeds within 15 to 50 h and the transformation of polymorphic Form 1 into polymorphic

Form 2 takes at least from 5 to 25 h. The term ‘crystallization time of polymorphic Form 1’ is defined as the time period required to obtain at least 70% of polymorphic Form 1, with the absence of polymorphic Form 2, since the clopidogrel base and sulfuric acid reaction was complete. The content of the polymorphic forms in the reaction mixture is monitored by the presence of the characteristic bands in IR spectra.

The present invention is achieved using the solvent system consisting of at least one solvent in which the transformations of crystal forms proceed very slowly, over 50 h; and at least one solvent in which the transformations are very fast. Due to the isolation of clopidogrel hydrogensulfate from the reaction mixture at the appropriate moment of phase transformation combined with a process of working up the separated crystals, the obtained polymorphic Form 1 of clopidogrel hydrogensulfate is stable and further transformation into polymorphic Form 2 is not observed. For the process for the preparation of substantially pure polymorphic Form 1 of clopidogrel hydrogensulfate is characteristic, that the reaction of optically active clopidogrel base, methyl(+)-(S)-α-(2-chlorophenyl)-4,5,6,7-tetrahydrothieno[3,2,-c]pyridin-5-acetate, with sulfuric acid is carried out in the mixture of at least two solvents, the first one chosen from group I, comprising aliphatic ethers, and the second one from group II, comprising ketones, esters of C₁-C₃ carboxylic acids and aliphatic alcohols C₁-C₄, primary, secondary and tertiary aliphatic alcohols C₁-C₄, then the resulting suspension is stirred until at least 70% of amorphous clopidogrel hydrogensulfate formed is transformed into polymorphic Form 1 of clopidogrel hydrogensulfate, and finally, the crystalline solid is isolated from the reaction mixture and subject to additional work-up procedure, aiming at the formation and stabilization of polymorphic Form 1.

In Table 1 crystallization time periods of the formation of polymorphic Forms 1 and 2 in the single solvents or solvent systems are given. In most of the experiments the starting materials were used at the same concentrations, ie. clopidogrel base at 67 g/L and sulfuric acid 10% wt. as the solution in one of the solvents mentioned.

TABLE 1
The kinetics of clopidogrel hydrogensulfate polymorphic
forms formation

Time of single polymorph
formation [h]

| | Polymorphic | Polymorphic |
|---|---|---|
| Solvent | Form 1 | Form 2 |
| MIBK | 20 | 30 |
| MTBE | >115 | >150 |
| ETBE | >170 | >220 |
| EA | 5.5 | 8 |
| Acetone | — | 1 |
| MIBK/MTBE (3:1) | 25 | 40 |
| MIBK/MTBE (1:1) | 35 | 60 |
| MIBK/EA (4:1) | 15 | 23 |
| MIBK/EA (1:4) | 7 | 12 |
| MTBE/EA (4:1) | 22 | 40 |
| ETBE/EA (4:1) | 25 | 50 |
| MIBK | 24 | 38 |
| (concentration of the | | |
| starting base 100 g/l) | | |
| MIBK | 16 | 20 |
| (concentration of the | | |
| starting base 33.3 g/l) | | |

In the preferred embodiment of the invention the solvent systems are chosen from among ketone/ether and ketone/ester mixtures. The preferred systems consist of isobutyl methyl ketone/tert-butyl methyl ether and isobutyl methyl ketone/ethyl acetate.

The most preferred systems consists of isobutyl methyl ketone/tert-butyl methyl ether at volume ratio from 4:1 to 1:4, preferably about 1:1. Another preferable solvent system is the one, consisting of isobutyl methyl ketone/ethyl acetate at volume ratio from 4:1 to 1:4, preferably about 1:1.

In the reaction of clopidogrel hydrogensulfate formation, either concentrated sulfuric acid or its solution in a single solvent or the mixture of solvents used as the reaction medium can be applied. When diluted sulfuric acid is added, the product of high purity is obtained, without affecting clopidogrel hydrogensulfate optical purity. Preferably, sulfuric acid solution is used at concentration from 0.5 to 85% wt., most preferably about 10% wt.

The starting optically active clopidogrel base, ie. methyl(+)-(S)-α-(2-chlorophenyl)-4,5,6,7-tetrahydrothieno[3,2,-c]pyridin-5-acetate, can be prepared according to one of the prior art methods. For example, it may be prepared according to the patent EP 0281459 B1 experimental procedure consisting in the separation of diastereoisomeric mixture of methyl α-(2-chlorophenyl)-4,5,6,7-tetrahydrothieno[3,2,-c]pyridin-5-acetate 10-camphorsulfonate and the proper S-(+)-clopidogrel isomer release with the base. In EP 0466569 B1 cycloaddition reaction of methyl 2-(2-chlorophenyl)-2-[2-(thienyl)ethylamino]-acetate in the presence of paraformaldehyde and formic acid is described. This is the example of enantioselective synthesis of methyl(+)-(S)-α-(2-chlorophenyl)-4,5,6,7-tetrahydrothieno[3,2,-c]pyridin-5-acetate. Any other known method can be used for the clopidogrel base preparation.

According to the present invention, clopidogrel free base is dissolved in a single solvent or the solvents mixture at the temperature 15-30° C., preferably at about 20° C. In the reaction mixture the starting base concentration is from about 0.03 to 0.4 mole per 1 L.

In one of the embodiments of the invention, the procedure is based on sulfuric acid or its solution dropwise addition into the base solution. In the alternate embodiment, the base solution is added to the sulfuric acid solution.

In both cases, the solutions are combined at the rate, which enables to maintain the reaction temperature within the range from 10 to 30° C.

While dropping, clopidogrel hydrogensulfate crystalline solid is precipitating out in abundance. When the reaction is complete, the crystalline suspension is being stirred at the same temperature range from 10 to 30° C., preferably at about 20° C. Neither heating nor cooling is necessary.

The suspension is stirred, until at least 70% of amorphous clopidogrel hydrogensulfate is transformed into polymorphic Form 1, then the precipitated solid is isolated from the reaction mixture by means of filtration or decantation, for instance.

In the preferred embodiment of the invention the suspension is stirred, until transformation of at least 80% of amorphous clopidogrel hydrogensulfate into polymorph 1 is determined. Additional work-up after the isolation of the product, following this procedure, comprises of washing up, and drying under the vacuum or in the air at temperature not higher than 60° C. from 12 to 48 h, for example for 24 h.

It has also been found, that clopidogrel hydrogensulfate polymorphic Form 1 containing considerable amount of amorphous salt (up to 30%), spontaneously transforms into substantially pure polymorph 1, when, after isolation from the reaction mixture, conditioning up to 30 days. In this embodiment, additional work-up of the isolated solid comprises washing and conditioning at the temperature from 20 to 60° C., during the period from 48 h to 30 days.

In the other embodiment of the invention, clopidogrel hydrogensulfate crystalline solid is conditioned in a single solvent or in the mixture of solvents chosen from group I.

The polymorphic form of the obtained clopidogrel hydrogensulfate is determined on the basis of infrared absorption bands and X-ray powder diffraction (XRPD).

Fourier Transformation Infrared (FT-IR) spectra were recorded using KBr pellets technique (with Perkin Elmer BX FT-IR spectrometer, measurement range 4000-400 cm⁻¹, in 4 cm⁻¹ resolution). FT-IR spectrum of the polymorphic form 1 significantly differs from polymorphic Form 2 FT-IR spectrum, which was revealed in the International Patent publication WO 99/65915. The former spectrum also differs from that one of amorphous form, published in the Polish Patent Application No. P-355814. In Table 2 most significant IR absorption bands are collected. They serve for identification and distinguishing of the clopidogrel hydrogensulfate polymorphic forms.

TABLE 2
FT-IR (KBr pellets) spectra. The comparison of
characteristic absorption bands of polymorphic Forms 1 and 2 and
amorphous form of clopidogrel hydrogensulfate.

| Form 1 | Form 2 | Amorphous form |
|---|---|---|
| [cm⁻¹] | [cm⁻¹] | [cm⁻¹] |
| 2986 (m) | 2956 (m) | 2958 (m) |
| 2954 (w) | | |
| 1175 (s, bb) | 1187 (s) | 1058 (s, bb) |
| 871 (m) | 882 (m) | 844 (s, bb) |
| 840 (m) | 868 (s) | |
| 766 (m) | 773 (m) | 762 (m) |
| 716 (m) | 725 (m) | 723 (m) |
| | | 709 (m) |
| 584 (s) | 589 (m) | 582 (s, bb) |
| | 568 (m) | |

w—weak intensity band,
m—medium intensity band,
s—strong intensity band,
bb—broad band

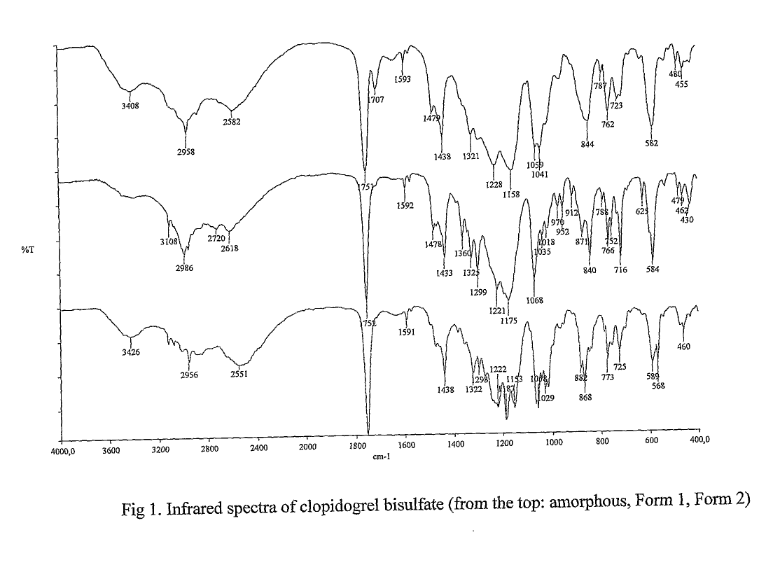

FIG. 1 presents the comparison of the FT-IR spectra in the full measurement range of the three following forms: clopidogrel hydrogensulfate polymorphic Form 1, obtained in the process of the present invention; polymorphic Form 2, data of which were revealed in WO 99/65915 and amorphous form of the clopidogrel salt.

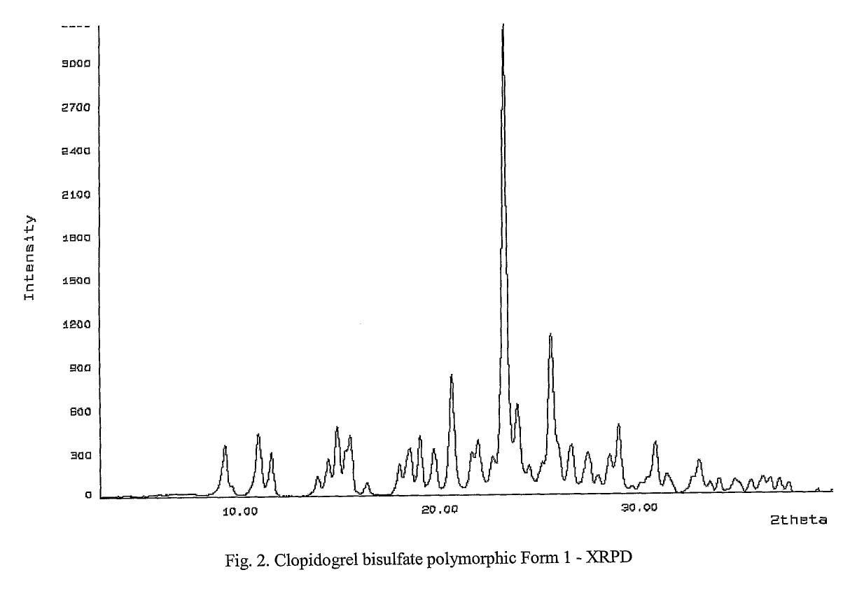

The standard X-ray powder diffraction pattern of clopidogrel hydrogensulfate polymorphpic Form 1 prepared by the method of the invention is depicted on FIG. 2. It was recorded with Rigaku MINI FLEX diffractometer using CuKα source. The result is presented as the relation of the diffraction lines relative intensities, diffraction angle 2θ and interplanar spacings d, in the range 3-40° in 2θ, scanning rate 0.5 deg/min and counting range 0.03 deg. The X-ray diffraction pattern is substantially similar to the one revealed in WO 99/65915.

Clopidogrel hydrogensulfate polymorphic Form 1, obtained in the process of present invention is of high optical and chemical purity. The content of the desired S enantiomer was determined by HPLC, it was 99.5%, usually higher than 99.8%. The content of the chemical impurities was less than 0.1%. Polymorphic Form 2 was undetectable.

In addition, obtained clopidogrel hydrogensulfate polymorphic Form 1 is stable under strass conditions. After 6 months storage at 40° C./RH 75% small increase of the impurities is observed.

The present invention is illustrated by the following examples, which should not be construed as any limitation of its scope.

### EXAMPLES

### Example I

Clopidogrel free base (10 g, 0.031 mole) is dissolved in 120 mL of tert-butyl methyl ether and ethyl acetate (3:1 v/v ratio) mixture and 1.73 mL (3.17 g, 0.031 mole) of concentrated sulfuric acid in 30 mL of tert-butyl methyl ether is added at temp. 20° C. The mixture is stirred at ambient temp. for 22 h. Product is filtered off, washed with tent-butyl methyl ether and dried under vacuum at temp. 40-50° C. for 24 h. 10.5 g of clopidogrel hydrogensulfate polymorphic Form 1 is obtained, which is confirmed by IR spectra. Optical purity 99.5%.

### Example II

Clopidogrel free base (20 g, 0.062 mole) is dissolved in 240 mL of isobutyl methyl ketone and ethyl acetate (3:1, v/v ratio) mixture. Then, 3.47 mL (6.34 g, 0.062) of concentrated sulfuric acid in 60 mL of isobutyl methyl ketone is added at temp. 20° C. and the solution is stirred at ambient temp. for 15 h. Product is filtered off, washed with isobutyl methyl ketone and dried under vacuum at temp. 40-50° C. for 24 h. Clopidogrel hydrogensulfate polymorphic Form 1 (22 g, 84.5%) is obtained, which is confirmed by IR spectra. Optical purity 99.5%.

### Example III

Clopidogrel free base (20 g, 0.062 mole) is dissolved in 240 mL of ethyl acetate and 3.47 mL (6.34 g, 0.062 mole) of concentrated sulfuric acid in 60 mL of isobutyl methyl ketone is added at temp. 20° C. The solution is stirred at ambient temp. for 7 h. Product is filtered off, washed with isobutyl methyl ketone and dried under vacuum at temp. 40-50° C. for 24 h. Clopidogrel hydrogensulfate polymorphic Form 1 (22 g, 84.5%) is obtained, which is confirmed by IR spectra. Optical purity 99.5%.

### Example IV

Clopidogrel free base (20 g, 0.062 mole) is dissolved in 240 mL of ethyl acetate and 3.47 mL (6.34 g, 0.062 mole) of concentrated sulfuric acid in 60 mL of ethyl acetate is added at temp. 20° C. The solution is stirred at ambient temp. for 5.5 h. Product is filtered off, washed with isobutyl methyl ketone and dried under vacuum at temp. 30-50° C. for 24 h. Clopidogrel hydrogensulfate polymorphic Form 1 (21.5 g, 82.6%) is obtained, which is confirmed by IR spectra. Optical purity 99.5%.

### Example V

Clopidogrel free base (20 g, 0.062 mole) is dissolved in 240 mL of ethyl acetate and 3.47 mL (6.34 g, 0.062 mole) of concentrated sulfuric acid in 60 mL of ethyl acetate is added at temp. 20° C. The solution is stirred at ambient temp. for 5.5 h. Product is filtered off, washed twice with tert-butyl methyl ether and dried under vacuum at temp. 30-50° C. for 24 h. Clopidogrel hydrogensulfate polymorphic Form 1 (21.4 g, 82.2%) is obtained, which is confirmed by IR spectra. Optical purity >>99.5%.

### Example VI

Clopidogrel free base (20 g, 0.062 mole) is dissolved in 240 mL of ethyl acetate and 3.47 mL (6.34 g, 0.062 mole) of concentrated sulfuric acid in 60 mL of ethyl acetate is added at temp. 20° C. The solution is stirred at ambient temp. for 5.5 h. Product is filtered off, washed twice with isobutyl methyl ketone and dried under vacuum at temp. 30-50° C. for 24 h. Clopidogrel hydrogensulfate polymorphic Form 1 (22.4 g, 85.3%) is obtained, which is confirmed by IR spectra. Optical purity >>99.5%.

### Example VII

Clopidogrel free base (20 g, 0.062 mole) is dissolved in 300 mL of ethyl acetate and 3.47 mL (6.34 g, 0.062 mole) of neat concentrated sulfuric acid is slowly added at temp. 20° C. The solution is stirred at ambient temp. for 5 h. Product is filtered off, washed with ethyl acetate and dried under vacuum at temp. 30-50° C. for 24 h. Clopidogrel hydrogensulfate polymorphic Form 1 (19.3 g, 74.1%) is obtained, which is confirmed by IR spectra. Optical purity 98.5%.

### Example VIII

Clopidogrel free base (20 g, 0.062 mole) is dissolved in 240 mL of n-butyl acetate and 3.47 mL (6.34 g, 0.062 mole) of concentrated sulfuric acid in 60 mL of n-butyl acetate is added at temp. 20° C. The solution is stirred at ambient temp. for 18 h. Product is filtered off, washed with n-butyl acetate and dried under vacuum at temp. 30-50° C. for 24 h. Clopidogrel hydrogensulfate polymorphic Form 1 (21.6 g, 83.0%) is obtained, which is confirmed by IR spectra. Optical purity 99.5%.

### Example IX

Clopidogrel free base (20 g, 0.062 mole) is dissolved in 240 mL of isobutyl methyl ketone and 3.47 mL (6.34 g, 0.062 mole) of concentrated sulfuric acid in 60 mL of isobutyl methyl ketone is added at temp. 20° C. The solution is stirred at ambient temp. for 20 h. Product is filtered off, washed with isobutyl methyl ketone and dried under vacuum at temp. 30-50° C. for 24 h. Clopidogrel hydrogensulfate polymorphic Form 1 (22.0 g, 84.5%) is obtained, which is confirmed by IR spectra. Optical purity >>99.5%.

### Example X

Clopidogrel free base (20 g, 0.062 mole) is dissolved in 240 mL of tert-butyl methyl ether and 3.47 mL (6.34 g, 0.062 mole) of concentrated sulfuric acid in 60 mL of tert-butyl methyl ether was added at temp. 20° C. The solution is stirred at ambient temp. for 115 h. Product is filtered off, washed with tert-butyl methyl ether and dried under vacuum at temp. 30-50° C. for 24 h. Clopidogrel hydrogensulfate (21.6 g, 83.0%) is obtained, containing about 70% wt. of polymorphic Form 1 and about 30% wt. of amorphous form. The content of both forms is determined by IR spectra. Optical purity 99.5%.

### Example XI

Clopidogrel free base (20 g, 0.062 mole) is dissolved in 150 mL of isobutyl methyl ketone and 3.47 mL (6.34 g, 0.062 mole) of concentrated sulfuric acid in 50 mL of isobutyl methyl ketone is added at temp. 20° C. The solution is stirred at ambient temp. for 24 h. Product is filtered off, washed with isobutyl methyl ketone and dried under vacuum at temp. 30-50° C. for 24 h. Clopidogrel hydrogensulfate polymorphic Form 1 (22.6 g, 86.8%) is obtained, which is confirmed by IR spectra. Optical purity 99.5%.

### Example XII

Clopidogrel free base (20 g, 0.062 mole) is dissolved in 480 mL of isobutyl methyl ketone and 3.47 mL (6.34 g, 0.062 mole) of concentrated sulfuric acid in 120 mL of isobutyl methyl ketone is added at temp. 20° C. The solution is stirred at ambient temp. for 16 h. Product is filtered off, washed with isobutyl methyl ketone and dried under vacuum at temp. 30-50° C. for 24 h. Clopidogrel hydrogensulfate polymorphic Form 1 (21.6 g, 83.0%) is obtained, which is confirmed by IR spectra. Optical purity >>99.5%.

### Example XIII

Clopidogrel free base (20 g, 0.062 mole) is dissolved in 240 mL of 2-butanol and 3.47 mL (6.34 g, 0.062 mole) of concentrated sulfuric acid in 60 mL of 2-butanol is added at temp. 20° C. The solution is stirred at ambient temp. for 22 h. Product is filtered off, washed with 2-butanol and dried under vacuum at temp. 30-50° C. for 24 h. Clopidogrel hydrogensulfate polymorphic Form 1 (20.6 g, 79.1%) is obtained, which is confirmed by IR spectra. Optical purity 99.5%.

### Example XIV

Clopidogrel free base (20 g, 0.062 mole) is dissolved in 240 mL of tert-butyl ethyl ether and 3.47 mL (6.34 g, 0.062 mole) of concentrated sulfuric acid in 60 mL of ethyl acetate is added at temp. 20° C. The solution is stirred at ambient temp. for 25 h. Product is filtered off, washed with tert-butyl ethyl ether and dried under vacuum at temp. 30-50° C. for 24 h. Clopidogrel hydrogensulfate polymorphic Form 1 (21.6 g, 83.0%) is obtained, which is confirmed by IR spectra. Optical purity 99.5%.

### Example XV

Clopidogrel hydrogensulfate obtained in Example X (ca. 30% wt. of amorphous form) was seasoned at temp. 40° C. for 30 days. Polymorphic Form 1 of clopidogrel hydrogensulfate was obtained (content of amorphous form <2%). Polymorphic Form 1 formation was confirmed by IR spectra. Optical purity 99.5%.

### Example XVI

Clopidogrel free base (20 g, 0.062 mole) is dissolved in 60 mL of ethyl acetate and the solution is added dropwise into the 3.47 mL (6.34 g, 0.062 mole) of concentrated sulfuric acid in 240 mL of tert-butyl ethyl ether, at temp. 20° C. The solution is stirred at ambient temp. for 25 h. Product is filtered off, washed with tert-butyl ethyl ether and dried under vacuum at temp. 30-50° C. for 24 h. Clopidogrel hydrogensulfate polymorphic Form 1 (21.6 g, 83.0%) is obtained, which is confirmed by IR spectra. Optical purity >>99.5%.

### Example XVII

Clopidogrel free base (20 g, 0.062 mole) is dissolved in the mixture consisting of 120 mL of tert-butyl ethyl ether and 120 mL of tert-butyl methyl ether. To this solution 3.47 mL (6.34 g, 0.062) of concentrated sulfuric acid in 60 mL of ethyl acetate is added at temp. 20° C. The reaction mixture is stirred at ambient temp. for 28 h. Product is filtered off, washed with tert-butyl methyl ether and dried under vacuum at temp. 30-50° C. for 24 h. Clopidogrel hydrogensulfate polymorphic Form 1 (21.6 g, 83.0%) is obtained, which is confirmed by IR spectra. Optical purity >99.5%.

1. A process for the preparation of substantially pure polymorphic Form 1 of clopidogrel hydrogensulfate, wherein the reaction of optically active clopidogrel base with sulfuric acid is carried out in the mixture of at least two solvents, the first one chosen from group I, comprising aliphatic ethers, and the second one from group II, comprising ketones, esters of C₁-C₃ carboxylic acids and aliphatic alcohols C₁-C₄, primary, secondary and tertiary aliphatic alcohols C₁-C₄, then the resulting suspension is stirred until at least 70% of amorphous clopidogrel hydrogensulfate formed is transformed into polymorphic Form 1 of clopidogrel hydrogensulfate, and finally, the crystalline solid is isolated from the reaction mixture and subject to additional work-up procedure, aiming at the formation and stabilization of polymorphic Form 1.

2. The process according to claim 1, wherein group I comprises tert-butyl methyl ether and tert-butyl ethyl ether, and group II comprises methyl isobutyl ketone, methyl isopropyl ketone, ethyl acetate, butyl acetate, isopropanol, n-butanol and 2-butanol.

3. The process according to claim 1, wherein either the solvents mixture of methyl isobutyl ketone/tert-butyl methyl ether or the mixture of methyl isobutyl ketone/ethyl acetate is used.

4. The process according to claim 3, wherein the solvents mixture of isobutyl methyl ketone/tert-butyl methyl ether is used at 4:1 to 1:4 volume ratio.

5. The process according to claim 4, wherein the solvents mixture of isobutyl methyl ketone/tert-butyl ethyl ether is used.

6. The process according to claim 5, wherein the solvents mixture of isobutyl methyl ketone/tert-butyl ethyl ether is used at 4:1 to 1:4 volume ratio.

7. The process according to claim 1, wherein the mixture of three solvents is used.

8. The process according to claim 1, wherein either concentrated sulfuric acid is used or its solution in a single solvent or the mixture of solvents, at the concentration from 0.5 to 85% wt.

9. The process according to claim 8, wherein the sulfuric acid is used as the solution at concentration from 5% to 15% wt.

10. The process according to claim 1, wherein concentrated sulfuric acid or its solution is added dropwise to the clopidogrel base solution.

11. The process according to claim 1, wherein the solution of clopidogrel base is added dropwise to sulfuric acid or its solution.

12. The process according to claim 1, wherein the suspension obtained upon the completion of the reaction of clopidogrel base with sulfuric acid is stirred, until more than 80% of the amorphous clopidogrel hydrogensulfate formed is transformed into polymorphic Form 1 of clopidogrel hydrogensulfate, which is subsequently isolated from the reaction mixture and subject to additional work-up procedure, comprising solid washing and drying either under or without vacuum, at the temperature range from 20 to 60° C., for the time period from about 12 to 48 h.

13. The process according to claim 1, wherein the suspension obtained upon the completion of the reaction of clopidogrel base with sulfuric acid is stirred, until at least 70% of amorphous clopidogrel hydrogensulfate formed is transformed into polymorphic Form 1 of clopidogrel hydrogensulfate, which is subsequently isolated from the reaction mixture and subject to additional work-up procedure, comprising solid washing and seasoning at temperature in the range from 20 to 60° C., for about 48 h up to 30 days.

14. The process according to claim 1, wherein the suspension is stirred, until at least 70% of amorphous clopidogrel hydrogensulfate formed is transformed into polymorphic Form 1 of clopidogrel hydrogensulfate, which is subsequently isolated from the reaction mixture and subject to additional work-up procedure, comprising solid washing and conditioning in a solvent chosen from group I.

15. Polymorphic Form 1 of clopidogrel hydrogensulfate prepared by the process according to any of the preceding claims, characterized by the optical purity more than 99.5% and total chemical impurities level less than 0.1%.

16. Polymorphic Form 1 of clopidogrel hydrogensulfate according to claim 15, characterized by optical purity more than 99.8% and total chemical impurities level less than 0.1%.

17. Polymorphic Form 1 of clopidogrel hydrogensulfate according to claim 15, characterized by the presence of none detectable amounts of other polymorphic forms.
